(19) United States
(12) Patent Application Publication
(10) Pub. No.: US 2009/0280517 A1
(43) Pub. Date: Nov. 12, 2009

(54) METHODS OF DIAGNOSIS AND PROGNOSIS FOR A MUSCULAR DYSTROPHY

(75) Inventors: Robert J. Bloch, Baltimore, MD (US); Patrick W. Reed, Rockville, MD (US)

Correspondence Address: MORGAN LEWIS & BOCKIUS LLP, 1111 PENNSYLVANIA AVENUE NW, WASHINGTON, DC, 20004, US

(73) Assignee: University of Maryland, Baltimore, MD (US)

(21) Appl. No.: 12/400,356

(22) Filed: Mar. 9, 2009

Related U.S. Application Data
(60) Provisional application No. 61/037,338, filed on Mar. 18, 2008.

(51) Int. Cl. C12Q 1/02

(52) U.S. Cl. 435/29

(57) ABSTRACT

The invention relates to the treatment, diagnosis, and prognosis of a muscular dystrophy or myopathy. The present inventors have found that the quantity of mu-crystallin is increased in a muscular dystrophy. In particular, the inventors have found that mu-crystallin is increased in facioscapulohumeral muscular dystrophy (FSHD). Based on the inventors' findings, the invention provides a novel means for the treatment, diagnosis, and prognosis of a muscular dystrophy or myopathy.

### RELATED APPLICATIONS

The present application is an application claiming the benefit pursuant to 35 U.S.C. § 119(e) of U.S. Provisional Patent-Application No. 61/037,338, filed Mar. 18, 2008. The entire disclosure and teachings of the above-referenced application is incorporated herein by reference.

### STATEMENT REGARDING FEDERALLY SPONSORED RESEARCH OR DEVELOPMENT

This invention was made with government support under NIH Grant No. R21 NS43976 awarded by the National Institutes of Health. The government has certain rights in the invention.

### TECHNICAL FIELD

The invention relates to diseases and disorders of muscle. More specifically, the invention relates to a muscular dystrophy or myopathy. In particular aspects, the invention relates to the treatment, diagnosis, and/or prognosis of a muscular dystrophy. In other particular aspects, the invention relates to the treatment, diagnosis, and/or prognosis of facioscapulohumeral muscular dystrophy.

### BACKGROUND OF INVENTION

Facioscapulohumeral muscular dystrophy (FSHD) is an inherited and progressive disease that preferentially affects muscles of the face, shoulder, and upper arms. Although it is primarily a disorder of skeletal muscle, FSHD is also associated with abnormalities of the retinal vasculature, sensorineural hearing defects and, in 12% of cases, cardiac arrhythmias (Am J Med Genet. 1985; 22:143-147; Acta Otolaryngol Suppl. 1995; 520 Pt 1:140-142; Muscle Nerve. 1995; 2:S73-S80; Eur Neurol. 2006; 56:1-5). With an estimated incidence of 1:15,000-1:20,000, there are ˜15,000-20,000 people in the United States of America, and close to 400,000 people worldwide suffering from FSHD. The normal life expectancy, the autosomal dominant mode of inheritance, and the high frequency of new mutations (˜10%) in FSHD patients all contribute to its rank as the third most common form of muscular dystrophy (Muscle Nerve. 2006; 34:1-15).

FSHD is thought to be genetically linked to deletions of integral numbers of 3.3 kb polymorphic repeating sequences from a tandem array, termed D4Z4, at chromosomal position 4q35. Detection of 10 or fewer repeats on chromosome 4 is diagnostic of the disease in over 95% of FSHD cases, but an additional requirement for disease pathogenesis is the inheritance of the 4qA allele on chromosome 4, indicating that a short D4Z4 array alone is not sufficient to cause FSHD (Nat. Genet. 2002; 32:235-236; Am J Hum Genet. 2004; 75:1124-1130). Although each 3.3 kb repeat contains a potential gene, DUX4 (double homeobox gene), no protein product of DUX4 has been found (Gene. 1999; 236:25-32). It has therefore been hypothesized that FSHD is caused by increasing the expression of genes at 4q35 through a positional effect (Chromosome Res. 1994; 2:225-234). This idea has recently been questioned, however (Hum Mol Genet. 2003; 12:2909-2921).

Several genes at 4q35 have been identified as potential targets of a possible positional effect, including FRG1, FRG2 (FSHD-Region Genes 1 and 2), TUBB4q pseudogene, PDLIM3, and ANT1. Tupler and her colleagues have produced evidence that up-regulation of cis genes occurs in FSHD as a result of a loss of binding sites for DNA-binding proteins in the D4Z4 repeats, and their subsequent binding to sites upstream of FRG1 and ANT1 (Cell. 2002; 110:339-348). In support of these results, they reported that over-expression of FRG1 in mice leads to muscular dystrophy, perhaps through aberrant splicing of mRNA (Nature. 2006; 439:973-977). These experiments are still controversial, however. In particular, careful analyses by several laboratories of muscle biopsies from patients with FSHD with gene arrays and quantitative RT-PCT fail to show an increase in the level of expression of ANT1 or FRG1 or indeed of any genes known to be cis to 4q35ter (see Muscle Nerve. 2006; 34:1-15). Although new genes are likely to be discovered in this region (e.g., a gene encoding a protein related to NMDA receptor), these results suggest that a broad increase in expression of the genes cis to the deletion at 4q35ter is unlikely to occur in FSHD Furthermore, the dystrophic phenotype is only seen in transgenic mice expressing a 30-40 fold increase in FRG1 compared to controls, with low dosages having little or no effect (Nature. 2006; 439:973-977; J. Physiol. 2007; 584:997-1009). Thus, high amounts of this protein may be needed for pathogenicity. However, it might be argued that these transgenic mice, like mdx mice, which lack dystrophin but live a normal life span (unlike patients with Duchenne muscular dystrophy, who are missing dystrophin and usually die in their twenties or earlier), simply resist the development of dystrophic symptoms, perhaps because of their small size (compared to man) and the fact that their muscles are subjected to much lighter loads during force generation. Nevertheless, the fact that FRG1 and other gene products cis to 4q35ter do not consistently show significant increases in FSHD argues that mechanisms other than a “position effect” should be investigated.

The same array studies that failed to identify, gene products that are consistently up-regulated in FSHD pointed instead to a role for changes in the mechanisms that control myogenic proliferation and differentiation, associated with the myogenic factor. MyoD, and the retinoblastoma protein, Rb (Brain. 2006; 129:996-1013). This is not consistent with in vitro studies that have shown that FSHD myoblasts proliferate and undergo early differentiation to form myotubes, which also occurs in controls, at least in vitro (Gene Ther. 2005; 12:1651-1662). Later stages in differentiation, including the expression of muscle-specific proteins and their assembly into sarcomeres and related structures, have not been assayed with FSHD muscle cells in vitro.

FSHD shares the changes in gene expression related to proliferation and differentiation with Emery-Dreifuss muscular dystrophy (EDMD), which is caused primarily by mutations of emerin or lamins, proteins of the inner nuclear membrane (Brain. 2006; 129:996-1013; Nat. Genet. 1994; 8:323-327; Nat. Genet. 1999; 21:285-288). Without being bound by theory, this may suggest that FSHD most closely resembles dystrophies linked to the nuclear envelope, and not dystrophies linked to the contractile apparatus or to the sarcolemma, despite the fact that the sarcolemma is altered in FSHD (Ann Neurol. 2006; 59:289-297). This conclusion is consistent with the association of 4q35ter with the nuclear envelope in normal myonuclei, though not with the persistence of this association in FSHD myonuclei, however (Hum Mol. Genet. 2004; 13:1857-1871; J. Cell Biol. 2004; 167:269-279). Whatever the underlying mechanism, the identity of the gene products associated with FSHD has remained elusive. As attempts to work from the level of the chromosome and gene expression have not led to specific candidate genes or proteins that are consistently altered in FSHD, the present inventors have studied the cellular and proteomic changes that occur in or during FSHD. The underlying assumption was that identifying these changes, and the structures and molecules involved, would yield important insights into the pathogenic mechanisms of FSHD, ultimately helping to explain how deletions at 4q35ter lead to disease and the potential for providing novel and unexpected methods of treating FSHD.

Despite decades of study, little is understood with regard to the mechanisms leading to muscle weakness and ultimately to the loss of muscle fibers that occurs in FSHD. Many forms of muscular dystrophy have been linked to mutations that disrupt sarcolemmal stability, either directly or indirectly. For example, in Duchenne muscular dystrophy (DMD), the primary mutation results in loss of dystrophin, a protein that is believed to protect the sarcolemma against damage caused by contraction. Other mutations can affect the sarcolemma indirectly, for example in Fukuyama muscular dystrophy and, as the present inventors and other have found, in the Large^{myd} mouse. In these diseases, the primary mutations affect an enzyme that is necessary for post-translational processing of the sarcolemmal protein, dystroglycan. This in turn is thought to disrupt the link between dystroglycan and its ligands in the muscle basal lamina.

Early microscopic studies suggested that the sarcolemma was altered in skeletal muscle from FSHD patients. Freeze fracture electron microscope revealed that the number of orthogonal arrays of intramembranous particles, now believed to be aquaporin complexes, is reduced in the plasma membrane of patients with FSHD, but also showed that they were reduced in patients with Duchenne muscular dystrophy, suggesting that the effect might be a non-specific effect of muscle disease (Acta Neuropathol. 1981:54:189-97; see also FASEB J. 2002; 16:1120-1122). Another study showed the presence of membrane attack complex on non-necrotic muscle fibers in FSHD muscle in the absence of immunoglobulin deposits, suggesting that the alternate complement pathway was activated by molecular changes on the muscle fiber surface (Neurology. 1998; 50:41-46). FSHD is characterized by the presence of inflammation in limited regions of the muscle, however, raising the possibility that this pathway might also be activated non-specifically. Serum creatine kinase levels are only moderately elevated in FSHD patients. This argues against massive contraction-induced damage to the sarcolemma, such as that seen in Duchenne muscular dystrophy, and suggests that sarcolemmal damage may occur gradually and at low levels, in keeping with the slow progression of the disease.

Based on the foregoing, it is clear that there is an unfulfilled need in the art concerning the understanding of mechanisms of muscular dystrophies (including, for example, FSHD), and other myopathies, and methods of treating, diagnosing, and prognosis for the same. In light of this, the present inventors have provided such insights into the mechanisms of muscular dystrophies (including, for example, FSHD), and other myopathies, and novel and unexpected methods of treating, diagnosing, and prognosing the same. In particular aspects of the invention, the inventors have shown, for the first time, that mu-crystallin (also known as NADPH-dependent thyroid hormone binding protein) is upregulated in FSHD, that mu-crystallin is cytotoxic in vivo, that mu-crystallin represents a novel marker for FSHD, and that mu-crystallin represents a novel therapeutic target for the treatment of FSHD. It is noted that although particular aspects of the invention may be drawn to FSHD, the invention is applicable to all muscular dystrophies and other myopathies characterized by, at least, an elevated or increased quantity of mu-crystallin when compared to a sample not affected by the muscular dystrophy.

### BRIEF SUMMARY OF INVENTION

The invention relates to the treatment, diagnosis, and/or prognosis of a muscular dystrophy.

In certain embodiments, the invention is drawn to a method of treating a muscular dystrophy or myopathy comprising administering to a subject in need thereof a therapeutically effective amount of a composition that decreases or inhibits a biological activity of mu-crystallin.

In other embodiments, the invention is drawn to a method of diagnosis for a muscular dystrophy or myopathy comprising analyzing the quantity of mu-crystallin in a sample.

In further other embodiments, the invention is drawn to a method of prognosis for a muscular dystrophy or myopathy comprising analyzing the quantity of mu-crystallin in a sample.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1. Organization of the beta-spectrin-based membrane cytoskeleton in human skeletal muscle. Longitudinal cryosections were cut from skeletal muscle biopsies and immunolabeled with anti-bets-spectrin antibodies following methods standard in the inventors' laboratory. Confocal images and enlarged regions (boxed areas, shown at two-fold higher magnification in the upper right panels) are shown. The major, repeating structural features at the sarcolemma were traced with Photoshop and are shown below each insert (lower right panels). In control deltoid muscle (A, E) and biceps muscles (C), beta-spectrin is restricted to thick, transverse domains of costameres overlying the Z-lines (arrows, thick lines in tracings) and thin transverse domains overlying M-lines (arrowheads, thin lines in tracings, control samples only) of the underlying contractile apparatus. Control muscles often show small links between the Z- and M-lines, possibly corresponding to the longitudinal domains observed in rodent muscle (C, thin arrow). In quadriceps muscle from a patient with Duchenne muscular dystrophy (B), beta-spectrin is organized into polygonal arrays that bear no clear relationship to the underlying contractile apparatus. Compared to control biceps (C) and deltoid (E) muscles. FSHD biceps (D) and deltoid (F) muscles show a partial loss of alignment of beta-spectrin with the underlying contractile structures. Immunolabeling for beta-spectrin is present at domains of the sarcolemma overlying Z-disks (arrows, thick lines in tracings), but it is not present at the domains overlying M-bands over much of the surface of the fiber. (A limited region with normal M- and Z-domains is circled in panel F). Instead, it concentrates in diagonal structures that traverse the thicker structures overlying Z-disks (arrowheads, thin lines in tracings, FSHD samples only). (Bars=5 microns).

FIG. 2. Increased gap between the sarcolemma and contractile apparatus in FSHD muscle. Longitudinal sections of fixed, control and FSHD biceps were processed for electron microscopy, following methods that are standard in the inventors' laboratory. (A) High magnification electron micrographs of control muscle show a tight association (small bracket, black arrow) between the most superficial myofibrils and the sarcolemma. Where subsarcolemmal structures such as lipid droplets (LD) are present in controls, the sarcolemmal closely follows their contours (white arrow). (B) In FSHD muscle, a significant gap (large bracket, black arrow) exists between the Z-lines of superficial myofibrils and the sarcolemma, preventing its close association with the underlying cellular structures. The sarcolemma does not follow the contours of the underlying structures (white arrow, panel B), consistent with a loss of tight links between the contractile apparatus and sarcolemma in FSHD muscle. The gaps between the most superficial Z-disk and the sarcolemma were measured in regions of control (C) and FSHD (D) biceps devoid of subsarcolemmal organelles that might artificially displace the sarcolemma. Bar=250 nm. (E) The average gap size in control muscle was 51.2±30.6 nm (62 measurements; 4 samples from 3 patients). The gap size in FSHD muscle was 210±103 nm (80 measurements; 5 patients), indicating a loss of the tight association between the contractile apparatus and sarcolemma in FSHD muscle. The difference was highly significant (p<0.001, Mann Whitney U test). Bars=mean values.

FIG. 3. Upregulation of mu-crystallin in FSHD seen on two-dimensional electrophoresis gels. Soluble proteins (100 μg) from 3 normal and 4 FSHD deltoid muscle were separated on Large-Gel 2D electrophoretic gels and stained with silver. Examples of 2 normals and 3 FSHD samples are shown. An arrow points to the spot apparent in FSHD but undetectable in extracts of control deltoid muscle. All other spots in these gels were represented in both control and FSHD samples.

FIG. 4. Immunoblotting of mu-crystallin in FSHD and other diseases of human muscle. Biopsy samples of patients with dermatomyositis (Derm: deltoid), polymyositis (Polymy: deltoid), Duchenne muscular dystrophy (DMD: quadriceps) and limb girdle muscular dystrophies 2B and 2I (LGMD2B and LGMD2I; quadriceps) were separated by SDS-PAGE and compared in immunoblots to some of the same control and FSHD biopsies shown in FIG. 3 (top panel). Protein loading was assessed with Ponceau Red (middle panel).

FIG. 5. Increased expression of human mu-crystallin in rat skeletal muscle in vivo causes a dystrophic phenotype. Tibialis anterior muscles in 4.5 week old rats were electroporated with 100 mg of plasmid encoding either GFP (pEGFP-c1) or a myc-epitope tagged form of mu-crystallin (pCMV-CRYM-myc), each of which are expressed under control of the CMV promoter. Rats were anesthetized, euthanized by perfusion-fixation, and their muscles removed, snap frozen and cryo-sectioned 4 weeks later. Sections were labeled with antibodies to GFP (left) or myc (right), followed by secondary antibodies coupled to Alexa-488. Controls showed that all labeling is specific. Nuclei were labeled with propidium iodide. Over-expression of GFP causes no pathology in skeletal muscle (left panel). By contrast, over-expression of mu-crystallin causes muscle pathology, indicated in the right panel by arrows pointing to: A, split fibers; B, central nuclei; C, necrosis; D, endomysial inflammation. Variability in the diameters of muscle fibers is also evident. Although this is an acute response, these features are also seen in FSHD.

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein in the claim(s), when used in conjunction with the word “comprising”, the words “a” or “an”, may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, an “agent” can be any molecular entity (including, for example, a small molecule, nucleic acid (such as, siRNA, shRNA expression cassette, antisense DNA, antisense RNA), protein, peptide, antibody, antisense drug, or other biomolecule that is naturally made, synthetically made, or semi-synthetically made) used alone or in combination with other molecular entities- or treatments that can alleviate, reduce, ameliorate, prevent, or maintain in a state of remission clinical symptoms or diagnostic markers associated a muscular dystrophy or myopathy.

As used herein, “biological activit” refers to a pharmacodynamic and\or pharmacokinetic property including, for example, molecular binding, receptor binding, non-receptor binding, binding of any of the foregoing and resultant biochemical or physiological effect, efficacy, bioavailability, absorption, distribution, metabolism, elimination, or a lack thereof of any of the foregoing (for example, molecular binding and resultant lack of biochemical or physiological effect). In particular embodiments of the invention, a biological activity refers to the pathogenic effect of mu-crystallin on myocytes.

As used herein, a “sample” refers typically to any type of material of biological origin that comprises, for example, a nucleic acid, protein, antibody, lipid, organelle, cell, intracellular or extracellular fluid, tissue, or an organ or isolated organ from a subject, including, for example, DNA, RNA, mitochondrion, nuclei, blood, plasma, serum, fecal matter, urine, semen, bone marrow, bile, spinal fluid, lymph fluid, samples of the skin, external secretions of the skin, respiratory, intestinal, genitourinary tracts, and other internal cavities, tears, saliva, milk, blood cell, muscle, organ, or other material of biological origin known by those of ordinary skill in the art.

A “therapeutically effective amount” or a “therapeutic effective amount” is an amount of an agent of the invention that alleviates, totally or partially, the pathophysiological effects of a muscular dystrophy or myopathy. A therapeutically effective amount or a therapeutic effective amount can also be an amount that is given prophylactically thereby inhibiting any pathophysiological effects of muscular dystrophy or myopathy. The amount will depend upon, for example, subject size, gender, magnitude of the associated condition or injury, and genetic or non-genetic factors associated individual pharmacokinetic or pharmacodynamic properties of the administered molecule of the invention. For a given subject in need thereof a therapeutically effective amount or a therapeutic effective amount can be determined by those of ordinary skill in the art and by methods known to those of ordinary skill in the art.

As used herein. “treat” and all its forms and tenses (including, for example, treat, treating, treated, and treatment) refer to both therapeutic treatment and prophylactic or preventative treatment. Those in need thereof, of treatment include those, already with a pathological condition of the invention (including, for example, a muscular dystrophy or myopathy) as well as those in which a pathological condition of the invention is to be prevented.

### II. The Present Invention

The present invention is drawn to novel methods of treating, diagnosing, and prognosing a muscular dystrophy or myopathy. In certain embodiments where the invention is drawn to a muscular dystrophy or other relevant embodiment, a muscular dystrophy of the present invention includes, for example, congenital muscular dystrophy, distal muscular dystrophy, Duchenne/Becker muscular dystrophy, Emery-Dreifuss muscular dystrophy, FSHD, a limb-girdle muscular dystrophy, multisystemic myotonic dystrophy, oculopharyngeal muscular dystrophy, scapuloperoneal muscular dystrophy, collagen disorders, and other muscular dystrophies related to the foregoing or known by those of ordinary skill in the art. In further embodiments where the invention is drawn a muscular dystrophy or other relevant embodiment, a muscular dystrophy is FSHD. In other certain embodiments where the invention is drawn to a myopathy or other relevant embodiment, a myopathy of the invention includes, for example, an inflammatory myopathy or myositis (including, for example, dermatomyositis, polymyositis, inclusion body myotosis), myotubular myopathy, nemaline myopathy, a desmin related myopathy, Marfan myopathy, a mitochondrial myopathy, and other myopathies related to the foregoing or known by those of ordinary skill in the art.

In other embodiments, the present invention is drawn to novel methods of treating, diagnosing, and prognosing a neuopathy, a retinal defect, or hearing loss.

The present inventors have found, and consequently were first to demonstrate, that mu-crystallin is upregulated in certain muscular dystrophies, in particular in FSHD. Without being bound by theory, the present inventors believe, which is supported by reasons, rationale, and data throughout the specification as would be readily known by one of ordinary skill in the art, that mu-crystallin is involved in other muscular dystrophies and myopathies, thereby representing a novel target for therapy, and a biological marker for diagnostic and prognostic methods comprising, for example, the detection of mu-crystallin. In support of the role of mu-crystallin in the pathophysiology of FSHD, its likely role as both a structural protein and an enzyme or binding protein for small ligands is important. This protein is also enriched in the lens of some animals and is concentrated in the mammalian retina; a tissue that is affected in a large number of FSHD patients, where it has been linked to retinopathy (Proc Natl Acad Sci USA. 1992, 89:9292-9296; Muscle Nerve 2, S73-S80; FASEB J. 19, 1683-1685), mu-Crystallin also has homology to enzymes involved in glutamate and ornithine metabolism and it binds NADPH, providing a potential link to the oxidative state of muscle, which appears to be altered in FSHD (Mol. Endocrinol. 11, 1728-1736). Also known as NADPH-dependent thyroid hormone binding protein, mu-crystallin has the potential to modulate the activity of T3, one of the most potent signaling molecules acting earl, in muscle differentiation to regulate myogenesis in cardiac and skeletal muscle, both of which are affected in FSHD patients (BioEssays 17, 211-218; Muscle Nerve 34, 1-15; Biochim. Biophys. Acta 1772, 186-194; Neuromuscul. Disord. 13, 322-333). Finally, mutations in mu-crystallin have been linked to deafness in man (Am J Hum Genet. 2003; 72:73-82; J. Med. Genet. 2006; 43: e25). Notably, FSHD patients commonly suffer hearing loss due to malfunctions of the inner ear (Muscle Nerve 2, S73-S80). Thus, mu-crystallin has been linked to most, if not all, of the biological processes known to be affected in FSHD.

In light of the upregulation of mu-crystallin and its pathological capacity, as found by the present inventors, in certain embodiments, the invention is drawn to treating a disease or condition of the instant invention, including, for example, FSHD, by administering a composition comprising an agent that decreases or inhibits the expression or a biological activity of mu-crystallin. Such agents include, for example, an antibody, an agent that causes RNA inference (including, for example, siRNA), a thyroid hormone binding protein, a thyroid hormone receptor agonist, partial agonist, or antagonist, a thyroid hormone (including, for example, triiodothyronine (T3) and thyroxine (T4)) and an NADPH binding protein.

In certain embodiments, the mechanisms of decreasing or increasing a biological activity of mu-crystallin related to thyroid hormone is predicated on mu-crystallin (also known as NADPH-dependent thyroid hormone binding protein), and its ability to bind T3, which is one of the most potent signaling molecules acting early in muscle differentiation to regulate myogenesis in cardiac and skeletal muscle, both of which are affected in FSHD patients (as discussed above).

In certain embodiments where the invention is drawn to an antibody or other relevant embodiment, an antibody refers to an immunoglobulin molecule that is able to specifically bind to a specific epitope on an antigen. In particular embodiments the antigen is present on or generated from mu-crystallin. An antibody of the present invention includes both therapeutic antibodies and diagnostic/prognostic antibodies. An antibody of the present invention can be an intact immunoglobulin derived from natural sources or from recombinant sources, and can be immunoreactive portions of an intact immunoglobulin (including, for example, an antibody fragment and a single chain antibody). An antibody is typically a tetramer of immunoglobulin molecules. An antibody of the present invention can be prepared by a variety of methods (Coligan et al. Current Protocols in Immunology (1991). For example, cells expressing a polypeptide of the present invention are administered to an animal to induce the production of sera containing polyclonal antibodies. In particular aspects, a preparation of the secreted protein is prepared and purified to render it substantially free of natural contaminants. Such a preparation is then introduced into an animal in order to produce polyclonal antisera of greater specific activity.

In particular embodiments, an antibody of the present invention is a monoclonal antibody (mAb), or protein binding fragment thereof. Such monoclonal antibody can be prepared, for example, using hybridoma technology (Nature 256:495 (1975); Eur. J. Immunol. 6:511 (1976); Eur. J. Immunol. 6:292 (1976); Monoclonal Antibodies and T-Cell Hybridomas, Elsevier, N.Y., pp. 563-681 (1981). In general, such methods involve immunizing an animal (e.g., a mouse) with polypeptide or with a secreted polypeptide-expressing cell. Such cells may be cultured in any suitable tissue culture medium; however, it may be preferable to culture cells in Earle's modified Eagyle's medium supplemented with 10% fetal bovine serum (inactivated at about 56.degree. C.), and supplemented with about 10 g/l of nonessential amino acids, about 1,000 U/ml of penicillin, and about 100 .mu.g/ml of streptomycin.

The splenocytes of, for example, such mice following the methods described above are extracted and fused with a suitable myeloma cell-line. Any suitable myeloma cell-line may be employed in accordance with the present invention; however, in particular embodiments the parent myeloma-cell-line (SP20), available from the ATCC is used. After fusion, the resulting hybridoma cells are selectively maintained in appropriate medium, and then cloned by limiting dilution as described by Wands et al. (Gastroenterology 80:225-232 (1981).) The hybridoma cells obtained through such a selection are then assayed to identify clones that secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated by one of ordinary skill in the art that Fab and F(ab′)2 and other fragments of the an antibody of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

In certain embodiments, for in vivo use of an antibody of the present invention in humans, it may be preferable to use a “humanized” chimeric monoclonal antibody, or other chimeric monoclonal antibody that does not illicit an immune response resulting from the antibody itself. Such an antibody can be produced using genetic constructs derived from hybridoma cells producing a monoclonal antibody as described above. Methods for producing a chimeric antibody are well known by one of ordinary skill in the art (see, for example, Science 229:1202 (1985); BioTechniques 4:214 (1986); U.S. Pat. No. 4,816,567; US Patent Application Publication No. 20060281095; EP 171496; EP 173494; WO 8601533; WO 8702671; Nature 312:643 (1984); Nature 314:268 (1985).

In certain embodiments where the invention is drawn to siRNA or other relevant embodiment, the invention comprises RNA inference (also referred to as “RNA-mediated interference”; RNAi). RNAi is a mechanism by which gene expression can be reduced or eliminated. Double stranded RNA (dsRNA) or single stranded RNA has been observed to mediate the reduction, which is a multi-step process (for details of single stranded RNA methods and compositions see, for example, Anal Biochem, 2002. 301(1): 103-10 and US Patent Application Publication No. 20070225241). dsRNA activates post-transcriptional gene expression surveillance mechanisms that appear to function to defend cells from virus infection and transposon activity (see, for example, Current Topics in Microbiology and Immunology: RNA Interference, Springer (2008); Fire et al., Nature, 391 (6669):806-811, 1998; Grishok et al., Science, 287:2494-2497, 2000; Ketting et al., Cell, 99 (2):133-141, 1999; Lin and Avery, Nature, 402:128-129, 1999, Montgomery et al., PNAS, 95:15502-15507, 1998; Sharp and Zamore, Science, 287:2431-2433, 2000; Tabara et al., Cell, 99 (2): 123-132, 1999; US Patent Application Publication No. 20070191262). Activation of these mechanisms targets mature, dsRNA-complementary mRNA for destruction. RNAi offers major experimental advantages for study of gene function and therapeutic silencing of genes. These advantages include a very high specificity, ease of movement across cell membranes, and prolonged down-regulation of the targeted gene. Moreover, dsRNA has been shown to silence genes in a wide range of systems, including plants, protozoans, fungi, C. elegans, Trypanasoma, Drosophila, and mammals (see, for example, Grishok et al., Science, 287:2494-2497, 2000; Sharp and Zamore, Science, 287:2431-2433, 2000; Sharp, Genes Dev., 13:139-141, 1999; Elbashir et al., Genes Dev. 5 (2):188-200. 2001; US Patent Application Publication No. 20070191262).

siRNAs are small RNAs that do not significantly induce the antiviral response common among vertebrate cells, but that do induce target mRNA degradation via the RNAi pathway. The term siRNA refers to RNA molecules that have either at least one double stranded region or at least one single stranded region and possess the ability to effect RNAi. It is contemplated that siRNA may refer to RNA molecules that have at least one double stranded region and possess the ability to effect RNAi. Mixtures or pools of dsRNAs (siRNAs) may be generated by various methods including chemical synthesis, enzymatic synthesis of multiple templates, digestion of long dsRNAs by a nuclease with RNAse III domains, and the like. A “pool” or “cocktail” refers to a composition that contains at least two siRNA molecules that have different selectivity with respect to each other, but are directed to the same target gene. Two or more siRNA molecules that have different selectivity with respect to each other, but are directed to the same or different target gene(s) are defined as different siRNAs. Different siRNAs may overlap in sequence, contain two sequences that are contiguous or non-contiguous in the target gene. In certain embodiments, a pool contains at least or at most 3, 4, 5, 6, 7, 8, 9, 10 or more siRNA molecules. An “siRNA directed to” a particular region or target gene means that a particular siRNA includes sequences that results in the reduction or elimination of expression of the target gene (i.e., the siRNA is targeted to the region or gene). The pool in some embodiments includes one or more control siRNA molecules. In other embodiments a control siRNA molecule is not included in the pool. A pool of siRNA molecules may also contain various candidate siRNA molecules that do not reduce or eliminate expression of a target gene.

Some of the uses for RNAi include implementing therapeutics and diagnostics, identifying genes that are essential for a particular biological pathway, identifying disease-causing genes, and studying structure-function relationships. As with other types of gene inhibitory compounds, such as antisense and triplex forming oligonucleotides, tracking these potential drugs in vivo and in vitro is important for drug development, pharmacokinetics, biodistribution, macro and microimaging metabolism, and for gaining a basic understanding of how these compounds behave and function. siRNAs have high specificity and may be used to knock out the expression of a single allele of a dominantly mutated diseased gene.

In certain embodiments of the invention, mu-crystallin is targeted with one or more siRNA molecules. Thus, in specific embodiments a muscular dystrophy or myopathy cell or a cell suspected of being or becoming susceptible to a muscular dystrophy or myopathy is provided with one or more siRNAs directed against mu-crystallin. In particular embodiments, an individual with a muscular dystrophy or myopathy, suspected of having a muscular dystrophy or myopathy, at a high risk for developing a muscular dystrophy or myopathy, or susceptible to a muscular dystrophy or myopathy, is administered one or more siRNA molecules directed against mu-crystallin. In particular embodiments where the invention is drawn to siRNA and a muscular dystrophy or other relevant embodiment, the siRNA is directed to mu-crystallin and a muscular dystrophy is FSHD.

The present invention includes methods and compositions for introducing multiple siRNAs targeting different regions of a gene that typically can greatly improve the likelihood that the expression of the target gene will be reduced. It is contemplated that candidate siRNAs or siRNAs do not interfere with the activities of others in the mixture and that in fact, there is some synergy between the siRNAs. This is applicable not only to siRNAs but to DNA constructs designed to express siRNAs (see, for example, Brummelkamp et al., Cancer Cell. 2002 September; 2(3):243-7: Brummelkamp et al., Science. 2002 Apr. 19; 296(5567):550-3). Certain embodiments of the invention alleviate the need to screen or optimize candidate siRNAs. In other embodiments, where this is not the case, to determine the functionality of a candidate siRNA or siRNA, it must be screened, verified, and/or optimized. As used herein, a “candidate siRNA” is an siRNA that has not been tested for its functionality as an siRNA. It is also contemplated that siRNAs may be single or double stranded RNA molecules.

In some embodiments of the invention, methods are employed wherein multiple therapeutic RNAs are employed, each of which reduce the expression of a target gene to some degree, as well as the presence of some dsRNAs, which do not effect target gene expression, may be administered as a pool without interference between members of the pool and may result in an additive or synergistic reduction in target gene expression. Thus, the present invention is directed to compositions and methods involving generation and utilization of pools or mixtures of, for example, small, double-stranded RNA molecules that effect, trigger, or induce RNAi more effectively. RNAi is mediated by an RNA-induced silencing complex (RISC), which associates (specifically binds one or more RISC components) with dsRNA pools of the invention and guides the dsRNA to its target mRNA through base-pairing interactions. Once the dsRNA is base-paired with its mRNA target, nucleases cleave the mRNA.

In certain embodiments of the invention, one or more siRNAs or dsRNAs can be introduced into a cell to activate the RNAi pathway. In other embodiments, various individual siRNAs or dsRNAs with different sequences may be co-transfected simultaneously to effectively produce a pool or mixture of dsRNAs within a transfected cell(s). The effects of multiple siRNAs are typically additive and may be synergistic in some cases.

In some embodiments, the invention concerns a siRNA or dsRNA that is capable of triggering RNA interference, a process by which a particular RNA sequence is destroyed (also referred to as gene silencing). siRNA are dsRNA molecules that are 100 bases or fewer in length (or have 100 basepairs or fewer in its complementarity region). A dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350, 375, 400, 450, 500, 550, 600, 650, 700, 750, 800, 850, 900, 950, or 1000 nucleotides or more in length. In certain embodiments, siRNA may be approximately 21 to 25 nucleotides in length. In some cases, it has about a two nucleotide 3′ overhang and a 5′ phosphate. The particular embodiments, an RNA sequence is targeted as a result of the complementarity between the dsRNA and the particular RNA sequence. It will be understood that siRNA or dsRNA of the invention can effect at least about a 20, 30, 40, 50, 60, 70, 80, 90 percent or more reduction of expression of a targeted RNA in a cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi comprise at least one region of complementarity within the RNA molecule. The complementary (also referred to as “complementarity”) region comprises at least or at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous bases. In some embodiments, long dsRNA are employed in which “long” refers to dsRNA that are 1000 bases or longer (or 1000 base pairs or longer in complementarity region). The term “dsRNA” includes “long dsRNA,” “intermediate dsRNA” or “small dsRNA” (lengths of 2 to 100 bases or basepairs in complementarity region) unless otherwise indicated.

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (e.g., in which a single strand with a hairpin loop is used as a dsRNA for RNAi). For convenience, lengths of dsRNA may be referred to in terms of bases, which simply refers to the length of a single strand or in terms of base pairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least about 50% complementary, and may be at least about 50%, 60%, 70%, 80%, or 90% complementary. In the range of about 50% to 70% complementarity, such sequences may be referred to as “very complementary,” while the range of greater than about 70% to less than complete complementarity can be referred to as “highly complementary.” Unless otherwise specified, sequences that are “complementary” include sequences that are “very complementary,” “highly complementary,” and “fully complementary.” It is also contemplated that any embodiment discussed herein with respect to “complementary” strands or region can be employed with specifically “fully complementary,” “highly complementary,” and/or “very complementary” strands or regions, and vice versa. Thus, it is contemplated that in some instances that siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability.

It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

The single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 or more (including the full-lehgth of a particular's gene's mRNA without the poly-A tail) bases or base pairs. If the dsRNA is comprised of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

Furthermore, it is contemplated that siRNA or the longer dsRNA template may be labeled. The label may be, for example, fluorescent, radioactive, enzymatic, or calorimetric. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C). When two or more differentially colored labels are employed, fluorescent resonance energy transfer (FRET) techniques may, for example, be employed to characterize the dsRNA.

Labels contemplated for use in several embodiments may be non-radioactive. In many embodiments of the invention, the labels are fluorescent, though they may be enzymatic, radioactive, or positron emitters. Fluorescent labels that may be used include, but are not limited to, BODIPY™, ALEXA FLUOR™, fluorescein, OREGON GREEN™, tetramethylrhodamine, TEXAS RED™ rhodamine, cyanine dye, or derivatives thereof. The labels may also, for example, more specifically be ALEXA 350™, ALEXA 430™, AMCA™, BODIPY 630/650™, BODIPY 650/665™, BODIPY-FL™, BODIPY-R6G™, BODIPY-TMR™, BODIPY-TRX™, CASCADE BLUE™, CY3™, CY5™, DAPI, 6-FAM, fluorescein isothiocyanate, HEX, 6-JOE. OREGON GREEN 488™, OREGON GREEN 500™, OREGON GREEN 514™, PACIFIC BLUE™, REG™, rhodamine green, rhodamine red, renographin, ROX™, SYPRO™, TAMRA™, TET™, tetramethylrhodamine, and/or TEXAS RED™. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as CY3™, CY5™, or fluorescein (FAM). In still further embodiments, the labeling reagent is also incubated with a labeling buffer, which may be any buffer compatible with physiological conditions and function (i.e.; buffers that is not toxic or harmful to a cell or cell component, termed “physiological buffer”).

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity, including hybridization, of the dsRNA. A person of ordinary skill in the art is well aware of achieving hybridization of complementary regions of molecules, such methods typically involve heat and slow cooling of temperature during incubation.

Any cell that undergoes RNAi can be employed in methods of the invention. The cell may be, for example, a eukaryotic cell, mammalian cell such as, for example, a primate, rodent, rabbit, canine, feline, equine, or human cell. In certain embodiments of the invention, there are methods of reducing the expression of a target gene in a cell. Such methods involve the compositions described herein. In various embodiments of the invention, reduction or elimination of expression of one or more target genes may be accomplished by a) obtaining one or more siRNA or dsRNA molecules corresponding one or more target genes and b) transfecting the respective siRNA or dsRNA molecules corresponding to the one or more target genes into a cell.

In other certain embodiments of the invention, siRNA and/or candidate siRNA molecules or template nucleic acids may be isolated or purified prior to their being used in a subsequent step, siRNA and/or candidate siRNA molecules may be isolated or purified prior to introduction into a cell. “Introduction” into a cell includes known methods of transfection, transduction, infection and other methods for introducing an expression vector or a heterologous nucleic acid into a cell. A template nucleic acid or amplification primer may be isolated or purified prior to it being transcribed or amplified. Isolation or purification can be performed by a number of methods known to those of ordinary skill in the art with respect to nucleic acids. In particular embodiments, a gel, such as an agarose or acrylamide gel, is employed to isolate the siRNA and/or candidate siRNA:

In other embodiments of the invention dsRNA is obtained by transcribing each strand of the dsRNA from one or more cDNA (or DNA or RNA) encoding the strands in vitro. It is contemplated that a single template nucleic acid molecule may be used to transcribe a single RNA strand that has at least one region of complementarity (and is thus double-stranded under conditions of hybridization) or it may be used to transcribe two separate complementary RNA molecules. Alternatively, more than one template nucleic acid molecule may be transcribed to generate two separate RNA strands that are complementary to one another and capable of forming a dsRNA. Additional methods involve isolating the transcribed strand(s) and/or incubating the strand(s) under conditions that allow the strand(s) to hybridize to their complementary strands (or regions if a single strand is employed).

In certain embodiments, nucleic acid templates may be generated by a number of methods well known to those of ordinary skill in the art. In some embodiments the template, such as a cDNA, is synthesized through amplification or it may be a nucleic acid segment in or from a plasmid that comprises the template. In other embodiments, siRNAs are encoded by expression constructs. The expression constructs may be obtained and introduced into a cell. Once introduced into the cell the expression construct is transcribed to produce various siRNAs. Expression constructs include nucleic acids that provide for the transcription of a particular nucleic acid. Expression constructs comprise plasmid DNA, linear expression elements, circular expression elements, viral or non-viral expression constructs, and the like, all of which are contemplated as being used in the compositions and methods of the present invention. In certain embodiments at least about 2, 3, 4, 5, 6, 7, 8, 9, 10, 15, 20, or more siRNA molecules are encoded by a single expression construct. Expression of the siRNA molecules may be independently controlled by at least about 1, 2, 3, 4, 5, 6, 7, 8, 9, 10 or more promoter elements. In certain embodiments, at least about 1, 2, 3, 4, 5, 6, 7, 8, 9, 10 or more expression constructs may introduced into the cell. Each expression construct may encode 1, 2, 3, 4, 5, 6, 7, 8, 9, 10, 15, 20 or more siRNA molecules. In certain embodiments siRNA molecules may be encoded as expression domains. Expression domains include a transcription control element, which may or may not be independent of other control or promoter elements; a nucleic acid encoding an siRNA; and optionally a transcriptional termination element. In other words, an siRNA cocktail or pool may be encoded by a single or multiple expression constructs. In particular embodiments the expression construct is a plasmid expression construct.

In other certain embodiments, the invention also concerns transcribing a strand or strands of a dsRNA using a promoter that can be employed in vitro or outside a cell, such as a prokaryotic promoter. In some embodiments, the prokaryotic promoter is a bacterial promoter or a bacteriophage promoter. In particular embodiments, it is contemplated that dsRNA strands are transcribed with SP6, T3, or T7 polymerase.

In certain embodiments where the invention is drawn to a use of a vector, which as used herein refers to a vehicle or other mechanism by which gene delivery or nucleic acid deliver can be accomplished, the invention encompasses delivery of a gene or nucleic acid for the purposes contemplated herein. In certain embodiments, gene delivery or nucleic acid delivery can be achieved by a number of mechanisms including, for example, vectors derived from viral and non-viral sources, cation complexes, nanoparticles (including, for example, ormosil and other nano-engineered, organically modified silica, and carbon nanotubes; see for example, Pantarotto et al., Chemistry&Biology. 2003; 10:961-966; 4ah et al., Mol. Therapy. 2000; 1:S239; Salata et al., J. Nanobiotechnology. 2004; 2:3) physical methods, bactofection, or any combination thereof.

In certain embodiments, the invention is, drawn to gene delivery or nucleic acid delivery comprising the use of viral vectors. Viruses are obligate intra-cellular parasites, designed through the course of evolution to infect cells, often with great specificity to a particular cell type. Viruses tend to be very efficient at transfecting their own DNA into the host cell, which is expressed to produce viral proteins. This characteristic and others, make viruses desirable and viable vectors for gene delivery or nucleic acid delivery. Viral vectors include both replication-competent and replication-defective vectors derived from various viruses. Viral vectors can be derived from a number of viruses, including, for example, polyoma virus, sindbis virus, fowlpox virus (UK 2,211,504 published 5 Jul. 1989), adenovirus and other viruses from the Adenoviridae family, adeno-associated virus and other viruses from the Parvoviridae family, herpes virus, vaccinia virus, alpha-virus, human immunodeficiency virus, papilloma virus, avian virus, cytomegalovirus, retrovirus, hepatitis-B virus, simian virus (including, for example, SV40), and chimeric viruses of any of the foregoing (including, for example, a chimeric adenovirus). Though a number of viral vectors can accomplish gene delivery or nucleic acid deliver, interest has concentrated on a finite number of viral vectors, including, for example, those derived from retrovirus, adenovirus, adeno-associated virus, and herpes virus. Examples of viral vectors include, for example, AAV-MCS (adeno-associated virus), AAV-MCS2 (adeno-associated virus), Ad-Cla (E1/E3 deleted adenovirus), Ad-BGFP-Cla (E1/E3 deleted adenovirus), Ad-TRE (E1/E3 deleted adenovirus). MMP (MPSV/MLV derived retrovirus), MMP-iresGFP (MPSV/MLV derived retrovirus), MMP-iresGFPneo (MPSV/MLV derived retrovirus), SFG-TRE-ECT3 (3′ Enhancer deleted, MLV derived retrovirus), SFG-TRE-IRTECT3 (3′ Enhancer deleted, MLV derived retrovirus). HRST (3′ Enhancer deleted HIV derived retrovirus), simian adenovirus and chimeric adenovirus (see, for example, US Patent Application Publication Nos. 20060211115, 20050069866, 20040241181, 20040171807, 20040136963, and 20030207259).

In other embodiments, gene delivery or nucleic acid delivery also includes vectors comprising polynucleotide complexes comprising cyclodextrin-containing polycations (CDPs), other cationic non-lipid complexes (polyplexes), and cationic lipids complexes (lipoplexes) as carriers for gene delivery or nucleic acid delivery, which condense nucleic acids into complexes suitable for cellular uptake (see, for example, U.S. Pat. No. 6,080,728; Liu et al. Current Medicinal Chemistry, 2003, 10, 1307-1315; Gonazalez et al., Bioconjugate Chemistry, 6:1068-1074 (1999); Hwang et al., Bioconjugate Chemistry 12:280-290 (2001)). A systems approach to prepare complexes and modify them with stabilizing and targeting components that result in stable, well-defined DNA- or RNA-containing complexes are suitable for in vivo administration. For example, polycations containing cyclodextrin can achieve high transfection efficiencies while remaining essentially non-toxic. A number of these complexes have been prepared that include variations in charge spacing, charge type, and sugar type (e.g., a spacing of six methylene units between adjacent amidine groups within the co-monomer gave the best transfection properties). Other polyplexes comprise, for example, polyethyleneimine (available from, for example, Avanti Lipids), polylysine (available from, for example, Sigma), polyhistidine (available from, for example, Sigma), and SUPERFECT (available from, for example, Qiagen) (cationic polymer carriers for gene delivery or nucleic acid delivery in vitro and in vivo has been described in the literature, see, for example, Goldman et al., Nature BioTechnology, 15:462 (1997)). Most polyplexes consist of cationic polymers and their complex production is regulated by ionic interactions. One large difference between the methods of action of polyplexes and lipoplexes is that some polyplexes cannot release their polynucleotides into the cytoplasm, which necessitates co-transfection with an endosome-lytic agent (to lyse the endosome that is made during endocytosis, the process by which a polyplex enters the cell) such as, for example, inactivated adenovirus. However this is not always the case, for example, polyplexes comprising polyethylenimine have their own method of endosome disruption, as does chitosan and trimethylchitosan.

Lipoplexes (also known as cationic liposomes) function similar to polyplexes and are complexes comprising positively charged lipids. Lipoplexes are increasingly being used in gene therapy due to their favorable interactions with negatively charged DNA and cell membranes, as well as due to their low toxicity. Due to the positive charge of cationic lipids they naturally complex with the negatively charged DNA. Also as a result of their charge they interact with the cell membrane, endocytosis of a lipoplex occurs and the polynucleotide of interest is released into the cytoplasm. The cationic lipids also protect against degradation of the polynucleotide by the cell. The use of cationic lipids for gene delivery or nucleic acid delivery was initiated by Felgner and colleagues in 1987, who reported that liposomes consisting of N-[1-(2,3-dioleyloxy) propyl]-N,N,N-trimethylammonium chloride (DOTMA) and dioleoylphosphatidylethanolamine (DOPE) were capable of facilitating effective polynucleotide transfer across cell membranes, resulting in high level expression of the encoded gene (Felgner et al., PNAS (1987) 84: 7413-7417). Since this seminal work, many new cationic lipids have been synthesized and have been shown to possess similar transfection activity, many of which are summarized by Balaban et al. (Expert Opinion on Therapeutic Patents (2001), 11(11): 1729-1752).

In other embodiments, gene delivery or nucleic acid delivery of the invention also includes vectors encompassing physical approaches for gene transfer into cells in vitro and in vivo (Gao et al., AAPS Journal. 2007; 9(1): E92-E104). Physical approaches induce transient injuries or defects in cell membranes so that DNA can enter the cells by diffusion. Gene delivery or nucleic acid delivery by physical approaches include, for example, needle injection of naked DNA (see, for example, Wolff et al., Science. 1990; 247:1465-1468), electroporation (see, for example, Heller et al., Expert Opin Drug Deliv. 2005; 2:255-268; Neumann et al., EMBO J. 1982; 1:841-845), gene gun (see, for example, Yang et al., PNAS 1990; 87:9568-9572; Yang et al., Nat Med. 1995; 1:481-483), ultrasound (see, for example, Lawrie et al., Gene Ther. 2000; 7:2023-2027), hydrodynamic delivery (see, for example, Liu et al., Gene Ther. 1999; 6:1258-1266; Zhang et al., Hum Gene Ther. 1999; 10:1735-1737), and laser-based energy (see, for example, Sagi et al., Prostate Cancer Prostatic Dis. 2003; 6(2):127-30).

In other embodiments, gene delivery or nucleic acid delivery of the invention also includes bactofection (see, for example, Palffy et al., Gene Ther. 2006 January; 13(2):101-5; Loessner et al., Expert Opin Biol Ther. 2004 February; 4(2): 157-68; Pilgrim et al., Gene Ther. 2003 November; 10(24):2036-45; Weiss et al., Curr Opin Biotechnol. 2001 October; 12(5):467-72; US Patent Application Publication No. 20030153527). Bacteria-mediated transfer of plasmid DNA into mammalian cells (i.e., bactofection) is a potent approach to introduce a gene or nucleic acid into a large set of different cell types in mammals. Applications include, for example, the expression of a therapeutic protein and RNAi. This mechanism of gene delivery or nucleic acid delivery uses bacteria for the direct transfer of nucleic acids into a target cell or cells. Transformed bacterial strains deliver the genes localized on plasmids into the cells, where these genes or nucleic acids are then expressed. Generally, the method of bactofection comprises using transformed invasive bacteria as a vector to transport genetic material, which is in the form of, for example, a plasmid comprising sequences needed for the transcription and translation of the protein of interest or the delivery of nucleic acids for the purpose of RNAi. For example, bactofection comprises the steps of: (a) transforming invasive bacteria to contain plasmids carrying the transgene; (b) the transformed bacteria penetrates into the cells; (c) vectors are destructed or undergo lysis, which is induced by the presence of the bacteria in the cytoplasm, and release plasmids carried; and (d) the released plasmids get into the nucleus whereupon the transgene is expressed. An analogous series of events transpire in the case of introducing nucleic acids for the purpose of RNAi, except in that case the nucleic acids decrease or inhibit the expression of one more proteins contemplated by the invention. Bacteria used in bactofection is preferably non-pathogenic or has a minimal pathogenic effect with said bacteria being either naturally occurring or genetically modified and is produced naturally, synthetically, or semi-synthetically. Bactofection has been reported with, for example, species of Shigella, Salmonella, Listeria, and Escherichia coli, with results suggesting that bactofection can be used with any bacterial species (Weiss et al., Curr Opin Biotechnol. 2001 October; 12(5):467-72).

In certain embodiments where the invention is drawn to a thyroid hormone binding protein or other relevant embodiment, a thyroid hormone binding protein includes, for example, a protein or other agent that binds mu-crystallin or inhibits the binding of a thyroid hormone to mu-crystallin thereby inhibiting mu-crystallin mediated pathophysiology.

In certain embodiments where the invention is drawn to a thyroid receptor agonist, partial agonist, or antagonist, or other relevant embodiment, a thyroid receptor agonist, partial agonist, or antagonist includes, for example, a protein or other agent that binds mu-crystallin or inhibits the binding of a thyroid hormone to mu-crystallin thereby inhibiting mu-crystallin mediated pathophysiology.

In certain embodiments where the invention is drawn to a thyroid hormone or other relevant embodiment, a thyroid hormone includes, for example, natural isolated thyroid hormone, synthetic recombinant thyroid hormone, a semi-synthetic thyroid hormone, liothyronine, levothyroxine, and liotrix. In particular embodiments the thyroid hormone is natural or synthetic T3, or an agonist of T3 receptors that produces a biological activity of T3.

In certain embodiments where the invention is drawn to an NADPH binding protein or other relevant embodiment, an NADPH binding protein includes, for example, a protein or other molecule that binds mu-crystallin or inhibits the binding of NADPH to mu-crystallin thereby inhibiting mu-crystallin mediated pathophysiology.

In certain embodiments where the invention is drawn to a method of diagnosis, prognosis or other relevant embodiment, an assay or assays are utilized for assessing the quantity of a biological marker, including, for example, mu-crystallin, in a sample to determine whether an individual is afflicted with a muscular dystrophy or myopathy, whether an individual's pathophysiology associated with a muscular dystrophy or myopathy is or has progressed, or whether an individual is at risk for (i.e., has a predisposition for or a susceptibility to) developing a muscular dystrophy or myopathy. A “biological marker” of the instant invention includes, for example, an endogenous molecule that can be measured in vitro, in vivo, ex vivo, or in situ, and that is associated with a muscular dystrophy or myopathy. In particular embodiments, a biological marker is mu-crystallin. In further particular embodiments, a muscular dystrophy is FSHD.

In certain embodiments, diagnosis or prognosis of a muscular dystrophy or myopathy comprises detecting the quantity of mu-crystallin nucleotides or polypeptides, and comparing that quantity to a control or other appropriate standard. A control or other appropriate standard is meant to refer to a baseline quantity of mu-crystallin that is utilized to determine a diagnosis or prognosis based on the analysis of mu-crystallin in a sample from a subject. For example, a baseline quantity of mu-crystallin can be obtained from a subject where a sample is obtained, wherein said sample is not affected by a muscular dystrophy or myopathy, which such baseline quantity of mu-crystallin may serve, for example, as a control or other appropriate standard for the diagnosis of a muscular dystrophy or myopathy. Also, for example, a baseline quantity of mu-crystallin can be obtained from a subject where a sample is obtained, wherein said sample is affected by a muscular dystrophy or myopathy, which such baseline quantity of mu-crystallin may serve, for example, as a control or other appropriate standard for the prognosis of a muscular dystrophy or myopathy. In particular embodiments, an increase in the quantity of mu-crystallin is important in determining the diagnosis or prognosis of a subject. For example, if a quantity of mu-crystallin is increased above control or other appropriate standard quantity of mu-crystallin, wherein the control or other appropriate standard was determined from a sample not affected by a muscular dystrophy or myopathy, then a diagnosis of a subject having a muscular dystrophy or myopathy is found. Also, for example, if a quantity of mu-crystallin is increased above control or other appropriate standard quantity of mu-crystallin, wherein the control or other appropriate standard was determined from a sample affected by a muscular dystrophy or myopathy, then a prognosis associated with a pathophysiology of a subject's muscular dystrophy or myopathy is found to be progressing or have progressed. Obtaining and using a control or other appropriate standard for the determination of a diagnosis or prognosis of the invention is well known by one of ordinary skill in the art. Therefore, examples recited herein are non-limiting and are meant for illustrative purposes only. In particular embodiments, a sample used for the diagnosis or prognosis of the invention is taken from a muscle.

In certain embodiments, a diagnosis or prognosis can be made by analyzing mu-crystallin polypeptide, by a variety of methods, including methods described herein, and also generally methods comprising spectroscopy, colorimetry, electrophoresis, isoelectric focusing, immunoprecipitations, and immunofluorescence, and immunoassays (e.g., David et al., U.S. Pat. No. 4,376,110) such as, for example immunoblotting (see also Current Protocols in Molecular Biology, particularly chapter 10). Both quantitative and qualitative increases of mu-crystallin are encompassed by the present invention. For example, in a particular embodiment, an antibody capable of binding to the polypeptide, preferably an antibody with a detectable label or an antibody that can be detected by a secondary antibody, can be used. Antibodies can be polyclonal or monoclonal. An intact antibody, or a fragment thereof (e.g., Fab or F (ab′) 2) can be used. The term “labeled” with regard to the probe or antibody, is intended to encompass direct labeling of the antibody by coupling (i.e., physically linking) a detectable substance to the antibody, as well as indirect labeling of the antibody by reactivity with another reagent that is directly labeled or indirectly labeled. Examples of direct and indirect labels include, for example, a fluorescent moiety, an enzyme, a chromophoric moiety, a radioactive atom, a biotin tag, or a colorimetric tag. Some examples of a fluorescent moiety include rhodamine, fluorescein, TEXAS RED™, etc. Some examples of enzymes include, horseradish peroxidase, glucose oxidase, glucose-6-phosphate dehydrogenase, alkaline phosphatase, beta-galactosidase, urease, luciferase, etc. Some examples of radioactive atoms are ³²P, ¹²⁵I, ³H, etc.

In other certain embodiments, the invention encompasses a kit comprising a reagent or composition as contemplated herein or as would be readily known by one of ordinary skill in the art for the treatment, diagnosis, or prognosis of a muscular dystrophy or myopathy. In other embodiments, the kit comprises a reagent or composition as contemplated herein or as would be readily known by one of ordinary skill in the art for the treatment, diagnosis, or prognosis of FSHD.

Reagents that are suited for obtaining a sample from an individual may be included in a kit of the invention, such as a syringe, collection vial, needle, or other instruments necessary to take a biopsy or other relevant sample.

The kits may comprise a suitably aliquoted composition and/or additional agent compositions of the present invention, whether labeled or unlabeled, as may be used to prepare a standard curve for a detection assay. The components of the kit may be packaged in combination or alone in the same or in separate containers, depending on, for example, cross-reactivity or stability, and can also be supplied in solid, liquid, lyophilized, or other applicable form. The container means of the kits will generally include, for example, at least one vial, test tube, flask, bottle, syringe or other container means, into which a component may be placed, and preferably, suitably aliquoted. Where there is more than one component in the kit, the kit can contain a second, third or other additional container into which the additional components may be contained. However, various combinations of components may be comprised in a vial. The kits of the present invention also will typically include a means for containing the composition, additional agent and any other reagent containers in close confinement for commercial sale. Such containers may include, for example, injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In this case, the container means may itself be a syringe, pipette, and/or other such like apparatus, from which the formulation may be applied to an infected area of the body, injected into an animal, and/or even applied to and/or mixed with the other components of the kit.

However, in other embodiments the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition is placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained. Irrespective of the number and/or type of containers, the kits of the invention may also comprise, and/or be packaged with, an instrument for assisting with the injection/administration and/or placement of a composition within the body of a subject or outside the body of a subject. Such an instrument may be a syringe, pipette, forceps, and/or any such medically approved delivery vehicle.

While the invention has been described with reference to certain embodiments, one of ordinary skill in the art appreciates that various modifications may be made without departing from the spirit and scope of the invention. Furthermore, the inventors note that the methods carried out as set forth throughout the specification may be modified or changed without departing from the spirit and scope of the invention. The scope of the appended claims is not to be limited to the specific embodiments described.

### Example 1

### Changes in Organization of the Sarcolemma in FSHD

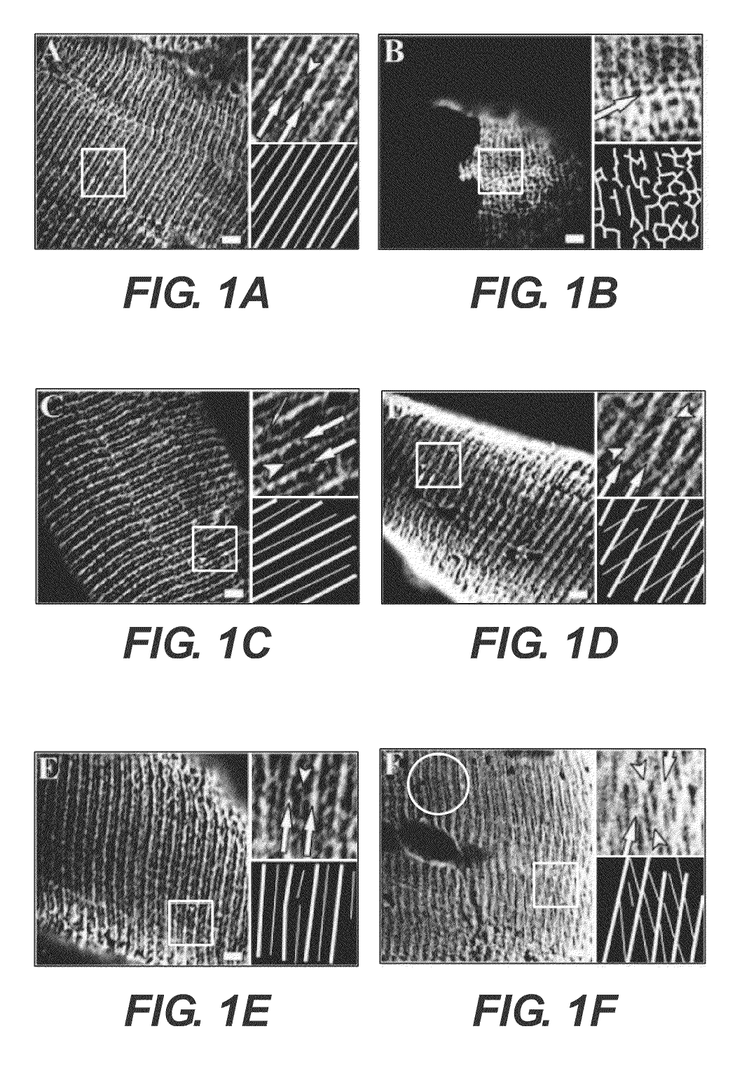

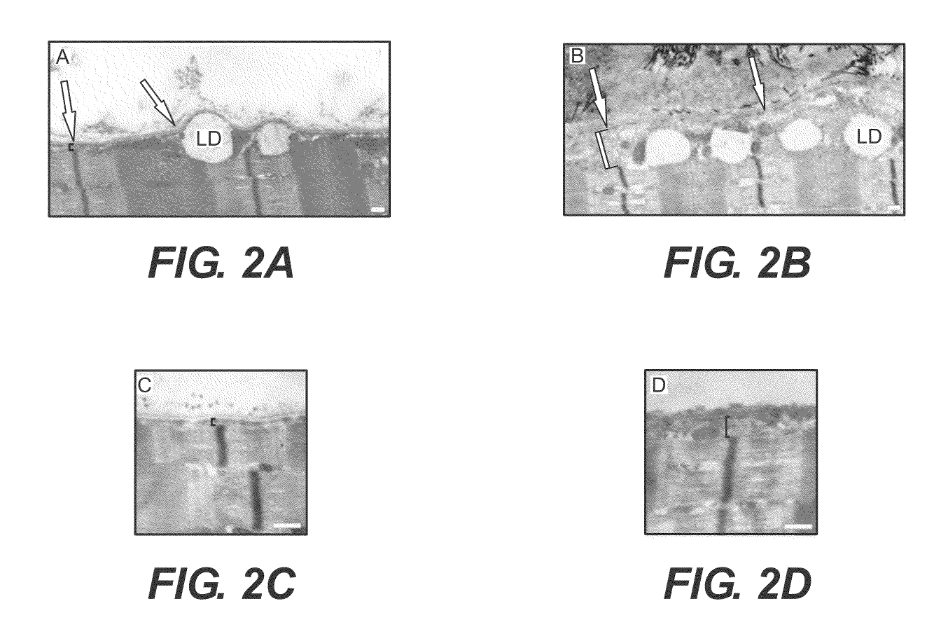

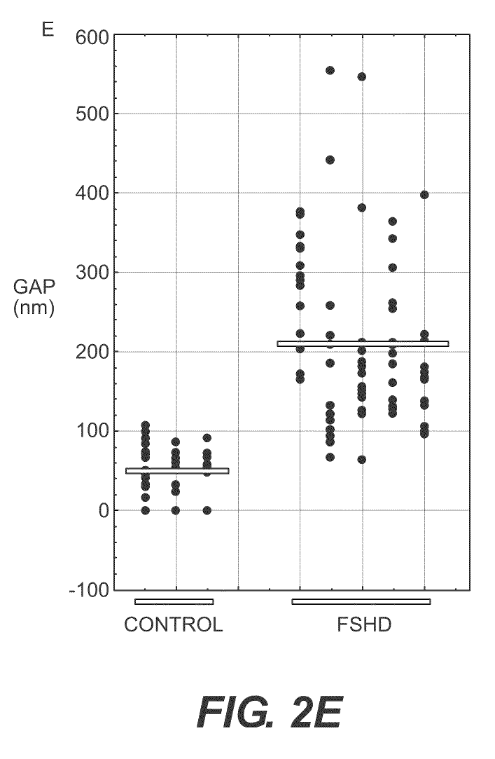

Methods were developed and used to examine longitudinal sections of unfixed human muscle biopsies, followed by immunofluorescent labeling of sarcolemmal proteins, to determine the organization of the sarcolemma and the subsarcolemmal membrane cytoskeleton in FSHD patient. Normally, the sarcolemma is organized into regular, transversely oriented membrane domains that overlie the Z-disks and M-bands of the superficial myofibrils. These structures have been called “costameres,” because of their “rib-like” appearance along the surface of each muscle fiber (Cell Motil. 1983: 3:449-462). Costameres are linked to nearby myofibrils at Z-disks and M-lines by at least 3 different kinds of cytoplasmic filaments, actin microfilaments, desmin-based intermediate filaments, and keratin-based intermediate filaments (Exerc Sport Sci Rev. 2003; 31:73-78; Clin Orthop Relat Res. 2002;S203-S210). They help to organize and stabilize the sarcolemma, harbor several signaling molecules, and are also involved in the transmission of contractile force laterally to the extracellular matrix and the tendon (Exerc Sport Sci Rev. 2003; 31:73-78). The present inventors found that FSHD muscle showed an unique reorganization of the subsarcolemmal membrane cytoskeleton and associated integral membrane proteins, in which distinct membrane domains were disoriented with respect to the underlying contractile apparatus (Ann Neurol. 2006; 59:289-297). Specifically, the domains of costameres, which in healthy muscle overlie M-bands, were oriented diagonally in FSHD (FIG. 1). Electron microscopy was also used to examine the relationship of the sarcolemma in glutaraldehyde-fixed skeletal muscle biopsies from patients with FSHD, which demonstrated a large increase (˜6-fold) in the distance between costameres at the sarcolemma and the nearest elements of the contractile apparatus in FSHD muscle, compared to controls (FIG. 2) (Ann Neurol. 2006; 59:289-297).

The above-discussed findings provide an important clue into the nature of some of the changes that occur in FSHD, namely that the sarcolemma and its relationship to nearby myofibrils is significantly altered. In addition, these findings can help explain, in part, the muscle weakness and the modest increase in serum creatine kinase that are typically seen in FSHD. As changes in “costameres” were implicated in the disease, presumably their assembly or the presence or modified composition of the proteins in these structures was altered, resulting in destabilization or a defective signaling. But costameres contain a number of different proteins, and are linked to the nearby contractile apparatus by three, presumably independent filamentous systems. Although progress has recently been made in studying the structure and development of costameres, little is understood about the protein-protein interactions that stabilize them, or the possible roles of chaperones in their assembly) and maintenance (Ann Neurol. 2006; 59:289-297). To this end, the inventors of the present invention have successfully undertaken research elucidating protein level differences found in FSHD patients.

### Example 2

### Protein Analysis FSHD

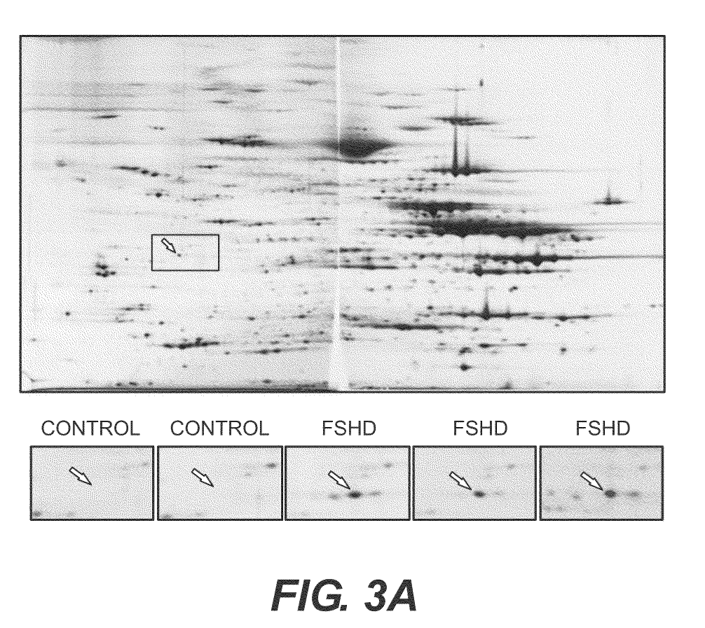

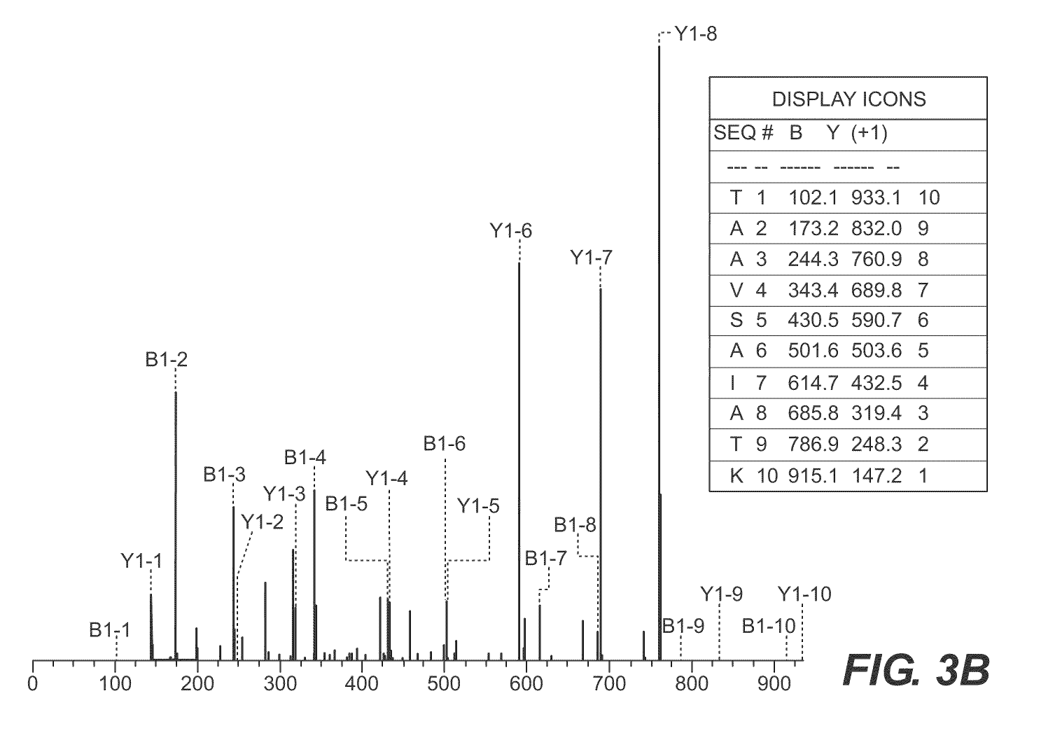

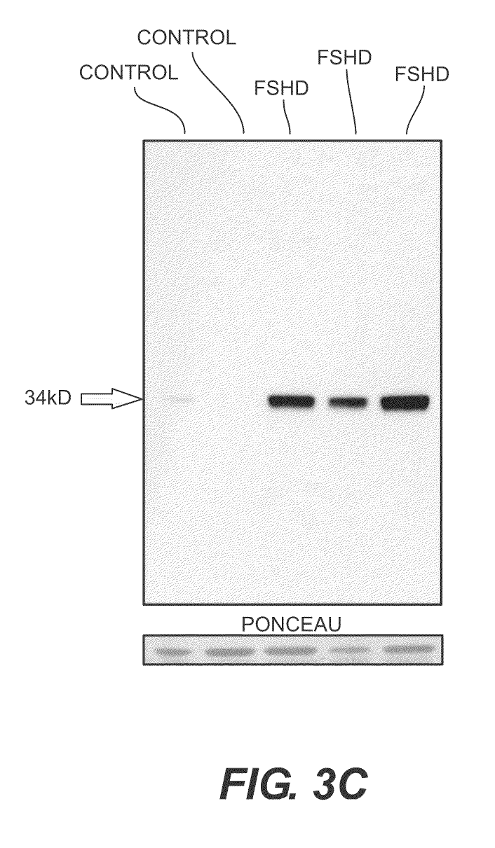

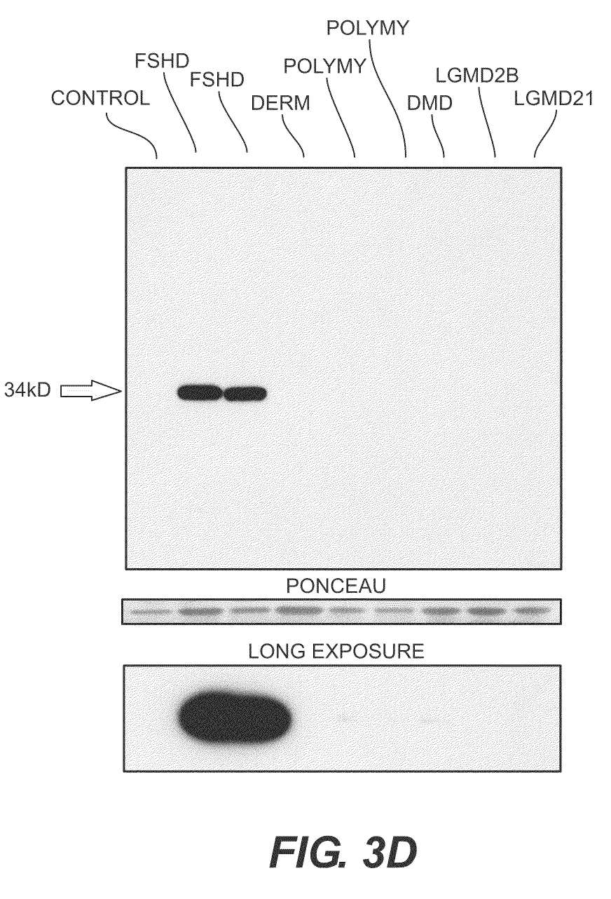

The present inventors examined proteomic changes associated with FSHD by studying open biopsies of, for example, deltoid muscles from FSHD and histologically normal control patients (FIG. 3). In an exemplary method, the biopsy samples were clamped at resting length, snap-frozen in isopentane, cooled with dry ice, and stored at −80° C. Soluble proteins were extracted from muscle biopsies as described. Two-dimensional electrophoresis (2DE) and silver staining were performed as described using 40 cm tube gels in the first dimension and two 20×20 cm2, 10% acrylamide slab gels, with some modifications (P. W. Reed and R. J. Bloch, manuscript in preparation) (Methods Mol. Biol. 112, 67-85; Methods Mol. Biol. 112, 147-172). Some of the modifications include, for example, (i) the use of 2 M thiourea, 7 M urea, instead of 9M urea, to dissolve samples prior to isoelectric focusing (IEF), to improve solubilization and inhibit proteases; (ii) the use of a strong reducing agent+vinylpyridine to reduce and then covalently modify cystine and cysteine residues, to prevent spurious disulfide bond formation; (iii) the use in IEF of narrow gauge polyacrylamide gels that have been preconditioned with the thiourea/urea solution, to reduce non-specific precipitation. (iv) digital enhancement of the images of the resultant gels, to reveal the presence of additional spots of proteins that are present in low amounts, which such modifications have yielded highly reproducible results with mammalian muscle and reveal >3,000 spots.

The inventors of the present invention used large format 2DE to search for proteins that are upregulated in FSHD. To maximize resolution, biopsies of deltoid muscle were separated into soluble, membrane, and insoluble fractions and analyzed individually. The soluble proteins were chosen first to be analyzed, and were extracted from the biopsies with Tris buffer (pH 7.1), without detergent. Visual comparison of the soluble protein fractions from control and FSHD deltoid showed only a few spots that changed clearly and consistently. As reported previously, albumin increased in FSHD samples compared to controls, but this may well have been due to serum contamination of the muscle biopsies (J. Mol. Med. 83, 216-224). Only one other spot was easily detected in all the FSHD samples we examined, but not in controls (FIGS. 3A and 3A′). This spot has an isoelectric point (pI) of 5.08 and apparent molecular mass of <34 kDa. Two spots flanking the major spot (FIG. 3A′) are likely to be charge variants of the same protein. None of these spots are present in samples of FSHD serum, suggesting that, unlike albumin, they derive from the muscle and not blood contaminants. Next, the spots were excised from a duplicate gel stained with Coomassie Blue, eluted with proteolysis, and subjected it to LC/MS/MS. Three peptides separated and sequenced by MS/MS corresponded to peptides of mu-crystallin a protein with a predicted pI and molecular mass matching the protein spot excised from the gel. These 3 peptides, TAAVSAIATK. FADTVQGEVR, and SLGMAVEDTVAAK, accounted for <10% of the mu-crystallin sequence and could not be matched to another protein. The identity of mu-crystallin was confirmed by immunoblotting (FIG. 3C). All FSHD samples showed a strong band at 34 kDa that labeled with antibodies to mu-crystallin; control samples did not show significant amounts of the protein. Non-immune controls failed to label a 34 kDa band, suggesting that the mu-crystallin band was specific. Labeling with Ponceau Red confirmed that the increased amount of mu-crystallin in FSHD samples was not due to differences in the amounts of protein analyzed, mu-Crystallin is therefore upregulated in the soluble fraction of deltoid muscles of FSHD patients. Without being bound by theory, the present inventors also believe that mu-crystallin is upregulated in the other muscles affected by FSHD, including for example, muscles of the face, other muscles of the shoulder, and muscles of the upper arms. In support of this, the present inventors have also find that there is increased quantities of mu-crystallin in the bicep muscles of FSHD patients.

The protein analysis of FSHD samples demonstrates that only one protein, mu-crystallin, is significantly altered in the soluble fraction, where it appears in large excess compared to controls, consistent with the autosomal dominant nature of the FSHD and other muscular dystrophies. Although mu-crystallin has not been identified as a protein linked to FSHD in previous studies and the gene that encodes it (CRYM) is not found near 4q35 (its human chromosomal locus is 16p13.11-p12.3), its known and potential biological activities make mu-crystallin a likely candidate involved in the pathogenesis observed in FSHD patients.

### Example 3

### Mu-Crystallin and Other Muscular Dystrophies

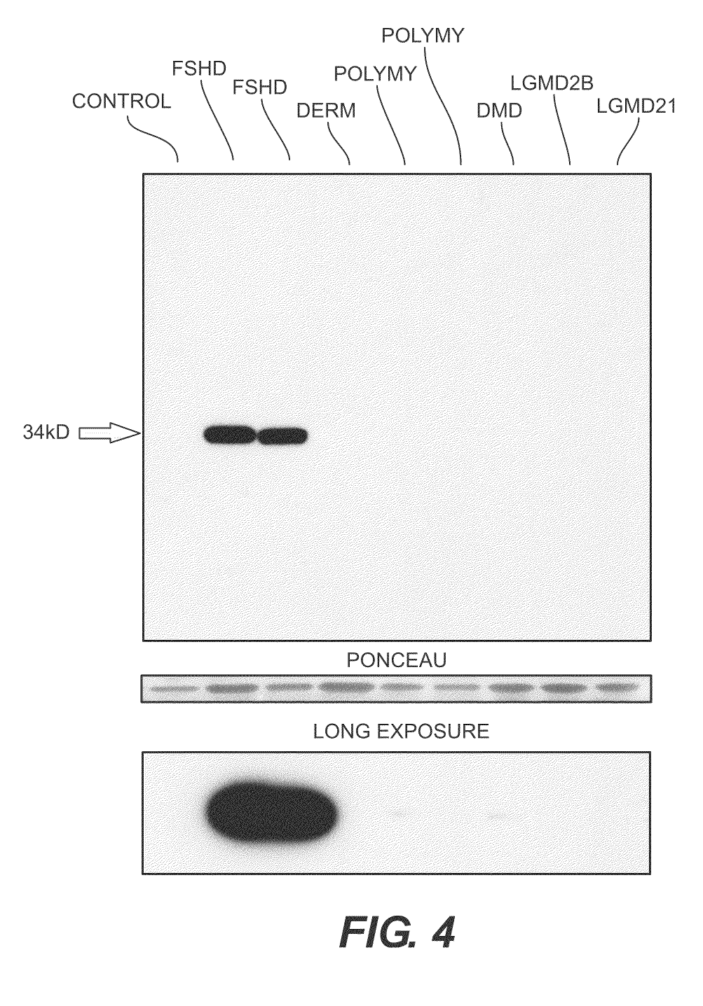

Immunoblots of soluble fractions prepared from biopsies of deltoid or quadriceps muscles of patients diagnosed with inflammatory myopathies, including dermatomyositis (deltoid) and, polymyositis (deltoid), and muscular dystrophies, including limb girdle muscular dystrophies types 2B and 2I (quadriceps), and Duchenne muscular dystrophy (quadriceps), showed no increases in mu-crystallin compared to controls (FIG. 4). However, the lack of increases in mu-crystallin found in these samples does not foreclose the possibilities that mu-crystallin is indeed upregulated in these other muscular dystrophies and myopathies. Without being bound by theory, the inventors of the present invention hold that based on mu-crystallin's upregulation in FSHD, and in vivo studies (discussed below), supports its role as a potential therapeutic, diagnostic, or prognostic for other muscular dystrophies and myopathies of the instant invention. For example, the inventors have observed an increased level of mu-crystallin in Marfan Myopathy, and array studies of MELAS mitochondrial myopathy also indicate the same (FASEB J. 2005 May; 19(7):866-8). A rationale as to why there may be no difference in mu-crystallin in the other muscular dystrophies analyzed is that the progression of the disease state may be at such a late stage as to show no discernable upregulation in mu-crystallin (i.e., the disease state may have progressed past a point where increased quantities of mu-crystallin are not detectable).

### Example 4

### Overexpression of Mu-Crystallin and In Vivo Pathology

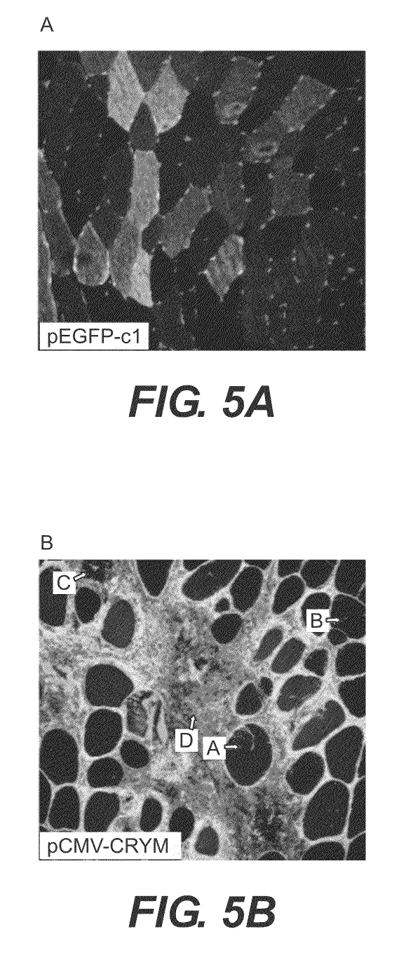

Overexpression of mu-crystallin in vivo induces muscle pathology. The overexpression of mu-crystallin was achieved by electroporation of plasmids encoding either EGFP or a myc-tagged human CRYM (mu-crystallin gene) under control of a CMV promoter fragment into the tibialis anterior muscles of 4 week old rats. Electroporation is an efficient method to induce expression in 30-50% of myofibers. When the muscles were examined at 4.5 weeks after electroporation, the EGFP muscles appeared normal (FIG. 5A). In contrast, muscles with overexpression of mu-crystallin showed significant pathological changes including split fibers, centrally nucleated fibers (indicating newly regenerated fibers), dead fibers with infiltrating cells, a large increase in fiber size variability, and a significant inflammatory response (FIG. 5B). It is not yet clear why much of the mu-crystallin appears to be outside of the myofibers. Among the possibilities are that it was released by dying fibers into the extracellular environment, that fibroblasts and satellite cells also took up the plasmid DNA and expressed it, or that, when overexpressed, mu-crystallin did not fold properly and was exported by the non-classical (i.e., not dependent on a signal sequence) secretory pathway. However, the acute pathological changes seen in these experiments, which are similar to those seen in FSHD, support the role of mu-crystallin in FSHD, and other muscular dystrophies and myopathies. These experiments have been repeated in preliminary studies with the rat sequence of mu-crystallin (CRYM), without the myc epitope tag, and support the conclusion that the endogenous protein itself, when overexpressed, is pathogenic.

### Example 5

### Overexpression of Mu-Crystallin Myoblasts and Myotubes

FSHD may be a disease linked to a failure of myoblasts to stop dividing and start differentiating, a process that is highly regulated by thyroid hormones, especially triiodothyronine (T3). Without being bound by theory, upregulation of mu-Crystallin may bind T3 thereby inhibiting certain myocyte differentiation. To this end, a tissue culture model by stably transfecting C2C12 myoblasts with cDNA encoding an epitope-tagged (e.g., myc, FLAG, HA) version of CRYM together with a cDNA encoding a version of GFP targeted to the nucleus is utilized. Establishment of stably transfected cell lines of C2C12 myoblasts is well described (see, e.g., Am J Physiol Regul Integr Comp Physiol. 2006; 290:R1672-R1682; J Cell Physiol. 2006; 207:428-436; Mol Endocrinol. 1992; 6:337-345). In brief, mu-crystallin is cloned with an appropriate epitope tag at its C-terminus, is inserted into, for example, pcDNA3, where its expression is under the control of the CMV promoter. Stably transfected myoblasts are selected with the antibiotic GENETICIN™, due to the resistance to this drug encoded by pcDNA3. Control cell-lines are similarly derived after transfection with empty plasmid. Several lines, expressing low, moderate and high amounts of mu-crystallin are established, to assess the effects of protein level. These cell-lines are assayed for proliferation (by, for example, measuring DNA with CYQUANT™ dye, as a function of days in culture), fusion (by, for example, determining % nuclei in myotubes) and differentiation (by, for example, expression of MyoD and other muscle-specific proteins [e.g., α-actin, skeletal myosin heavy chain]). Without being bound by theory, if T3 signaling is disrupted by mu-crystallin overexpression, then stably transfected myoblasts will fuse to form myotubes less than untransfected myoblasts or control cell lines. In this regard, addition of excess T3 to the culture medium (basal levels of T3 in the medium, which contains 10% fetal bovine serum, are normally sufficient to support muscle differentiation) overcomes this inhibition.

Mu-Crystallin over-expression in primary cultures of rat myotubes is also studied, using, for example, techniques to transfect cells biolistically. Transfection efficiencies are ˜50% (Sci STKE. 2003; 2003:PL 11). Samples are, for example, collected 1-3 days after transfection, are dissolved in SDS-PAGE sample buffer, are separated by SDS-PAGE and either stained with Coomassie Blue or transferred to nitrocellulose and blotted with antibodies to for example, MyoD following procedures standard to those of ordinary skill in the art (see, for example. J Cell Sci. 2001; 114:751-762). Alternatively or supplementary to the above methods, quantitative or semi-quantitative RT-PCR is performed to measure muscle-specific transcription. Without being bound by theory, if overexpression of mu-crystallin inhibits differentiation, the levels of proteins or their mRNAs related to differentiation (including, for example. MyoD) is significantly lower in overexpressors than in controls. Again, T3 may reverse this inhibition but only if overexpression of mu-crystallin has no other activity related to muscle differentiation.

This research conducted herein was supported under NIH Grant No. R21 NS43976 awarded by the National Institutes of Health. Grant No. FSHS-MGBF-014 awarded by the FSH Society, and Grant No. MDA 4359 awarded by the Muscular Dystrophy Association.

### REFERENCES

All patents and publications mentioned in this specification are indicative of the level of those skilled in the art to which the invention pertains. All patents and publications herein are incorporated by reference to the same extent as if each individual publication was specifically and individually indicated as having been incorporated by reference in its entirety.

What is claimed:

1. A method of diagnosis of a muscular dystrophy or myopathy comprising the steps of:
obtaining a sample from a subject suspected of having a muscular dystrophy or myopathy;
analyzing said sample for the presence of a biological marker; and
comparing the quantity of said biological marker to that of a control sample from said subject suspected of having a muscular dystrophy or myopathy or a control sample from a subject known not to have a muscular dystrophy or myopathy,
wherein if the quantity of the biological marker from said subject suspected of have a muscular dystrophy or myopathy is increased compared to the control said subject suspected of having a muscular dystrophy or myopathy is diagnosed as having a muscular dystrophy or myopathy.

2. The method of claim 1, wherein said obtaining a sample from a subject suspected of having a muscular dystrophy or myopathy is obtained from muscle affected by the muscular dystrophy or myopathy.

3. The method of claim 1, wherein said control sample from said subject suspected of having a muscular dystrophy or myopathy is obtained from muscle not affected by the muscular dystrophy or myopathy.

4. The method of claim 1, wherein said control sample from a subject known not to have a muscular dystrophy or myopathy is obtained from muscle.

5. The method of claim 1, wherein said samples are taken from the same muscle of said subject.

6. The method of claim 1, wherein said method is a method of diagnosis of a muscular dystrophy wherein said muscular dystrophy is FSHD.

7. The method of claim 1, wherein a myopathy is selected from the group consisting of an inflammatory myopathy or myositis, myotubular myopathy, nemaline myopathy, a desmin related myopathy, Marfan myopathy, a mitochondrial myopathy, and other myopathies.

8. The method of claim 1, wherein the biological marker is mu-crystallin.

9. A method of prognosis of a muscular dystrophy or myopathy comprising the steps of:
obtaining a sample from a subject that has been diagnosed as having a muscular dystrophy or myopathy;
analyzing said sample for the presence of a biological marker; and
comparing the quantity of said biological marker to that of a previous sample taken and analyzed for the presence of said biological marker from said subject diagnosed as having a muscular dystrophy or myopathy.
wherein if the quantity of the biological marker from said subject is increased compared to said previous sample said subject's prognosis is that said muscular dystrophy or myopathy is progressing pathologically.

10. The method of claim 9, wherein said obtaining a sample from said subject is obtained from muscle affected by the muscular dystrophy or myopathy.

11. The method of claim 9, wherein said previous sample taken and analyzed from said subject is obtained from muscle affected by the muscular dystrophy or myopathy.

12. The method of claim 9, wherein said samples are taken from the same muscle of said subject.

13. The method of claim 9, wherein said method is a method of prognosis of a muscular dystrophy wherein said muscular dystrophy is FSHD.

14. The method of claim 9, wherein a myopathy is selected from the group consisting of an inflammatory myopathy or myositis, myotubular myopathy, nemaline myopathy, a desmin related myopathy, Marfan myopathy, a mitochondrial myopathy, and other myopathies.

15. The method of claim 9, wherein the biological marker is mu-crystallin.
